(19) United States
(12) Patent Application Publication
(10) Pub. No.: US 2013/0273176 A1
(43) Pub. Date: Oct. 17, 2013

(54) METHODS AND COMPOSITIONS USEFUL FOR PROMOTING SLEEP IN ANIMALS

(71) Applicant: Brian Michael Zanghi, Ballwin, MO (US)

(72) Inventor: Brian Michael Zanghi, Ballwin, MO (US)

(73) Assignee: Nestec SA, Vevey (CH)

(21) Appl. No.: 13/991,716

(22) PCT Filed: Nov. 14, 2011
(86) PCT No.: PCT/US11/60597

§ 371 Date: Jun. 5, 2013

Related U.S. Application Data
(60) Provisional application No. 61/459,181, filed on Dec. 7, 2010.

(51) Int. Cl. A61K 31/4045; A61K 33/30; A61K 31/122

(52) U.S. Cl. CPC A61K 31/4045; A61K 31/122; A61K 33/30
USPC 424/641; 514/419

(57) ABSTRACT

Methods and compositions for promoting sleep in an animal are provided. In a general aspect, a sleep promoting amount of astaxanthin and a sleep promoting amount of melatonin are administered in conjunction to an animal. The astaxanthin can be administered in an amount ranging from about 0.1 to about 60 mg/kg/body weight of the animal. The melatonin can be administered in an amount ranging from about U. I to about 40 mg/kg/body weight of the animal. The method can further comprise administering in conjunction a sleep promoting amount of zinc to the animal. The zinc can be administered in an amount ranging from about 10 to about 100 mg/kg/body weight of the animal.

### CROSS REFERENCE TO RELATED APPLICATIONS

This application claims priority to U.S. Provisional Application Ser. No. 61/459,181 filed Dec. 7, 2010, the disclosure of which is incorporated herein by this reference.

### BACKGROUND OP THE INVENTION

1. Field of the Invention

The invention relates generally to methods and compositions useful for promoting sleep and particularly to the use of astaxanthin and melatonin to promote sleep in animals.

2. Description of Related Art

The sleep-wake cycle in animals is the most easily observable circadian biorhythm, and is an indispensable function of a healthy life. Normal aging is accompanied by an increased prevalence in sleep disturbances and decrease in sleep quality. As many as 80% of elderly subjects experience difficulty initialing sleep and also show increased night-time wakefulness, early waking, and/or increased daytime napping. Likewise, in dogs, aging has been associated with changes in sleep patterns and declining activity levels, as commonly reported by pet owners and observed under controlled conditions.

Increased sleep disturbances or decreased sleep can have deleterious effects, such as decreased cognitive performance like reduced mental acuity and reduced memory, decreased motor skills, and also increased health risks associated with depression, cardiovascular disease, obesity, and/or diabetes.

In people with sleep disturbances, medications are prescribed, but this may come with negative side effects like excessive sedation sensation or allergic reactions. Supplementation with purified melatonin has been used to promote sleep in people considered “normal” sleepers or those with certain types of sleep disorders. In some cases, purified melatonin improved sleep efficiency or reduced sleep onset time. However, this benefit has not been established in animals, like cats and dogs. Therefore, it is unclear if purified melatonin would have the same benefits in companion animals, as it does in people. It is also unclear if a combination of melatonin and other nutrients would promote the same sleep effects compared to melatonin alone. As a result, sleep aids for animals are generally non-existent. There is, therefore, a need for a composition capable of promoting sleep in animals.

### SUMMARY OF THE INVENTION

It is, therefore, an object of the present invention to provide methods for promoting sleep in animals.

It is another object of the invention to provide compositions useful for promoting sleep in senior animals.

It is a further object of the invention to promote the health or wellness of an animal.

It is yet another object of the present invention to improve the quality of life of an animal.

It is still another object of the present invention to extend the prime years of an animal's life.

One or more of these or other objects are achieved by administering in conjunction a sleep promoting amount of astaxanthin and a sleep promoting amount of melatonin to an animal. Generally, the astaxanthin is administered in an amount ranging from about 0.1 to about 60 mg/kg/body weight of the animal (mg/kg/bw) and the melatonin is administered in an amount ranging from about 0.1 to about 40 mg/kg/bw. The method can further comprise administering in conjunction a sleep promoting amount of zinc to the animal. The zinc is administered in an amount ranging from about 10 to about 100 mg/kg/bw.

Other and further objects, features, and advantages of the present invention will be readily apparent to those skilled in the art.

### DETAILED DESCRIPTION OF THE INVENTION

### Definitions

The term “animal” means a mammal capable of sleeping and benefiting from a sleep promoting composition. For example, animals can refer to pets such as dogs or cats or other mammals such as humans.

The term “in conjunction” means that astaxanthin, zinc, melatonin, or other compounds or compositions of the present invention are administered to an animal (1) together in a composition or (2) separately at the same or different frequency using the same or different administration routes at about the same time or periodically. “Periodically” means that the compound or composition is administered on a dosage schedule acceptable for a specific compound or composition. “About the same time” generally means that the compounds or compositions are administered at the same time or within about 4 hours of each other.

The term “single package” means that the components of a kit are physically associated in or with one or more containers and considered a unit for manufacture, distribution, sale, or use. Containers include, but are not limited to, bags, boxes, canons, bottles, packages of any type or design or material, over-wrap, shrink-wrap, affixed components (e.g., stapled, adhered, or the like), or combinations thereof. A single package may be containers of individual components physically associated such that they are considered a unit for manufacture, distribution, sale, or use.

The term “virtual package” means that the components of a kit are associated by directions on one or more physical or virtual kit components instructing the user how to obtain the other components, e.g., a bag or other container containing one component and directions instructing the user to go to a website, contact a recorded message or a fax-back service, view a visual message, or contact a caregiver or instructor to obtain instructions on how to use the kit or safety or technical information about one or more components of a kit.

The term “extending the prime” means extending the number of years an animal lives a healthy life and not just extending the number of years an animal lives, e.g., an animal would be healthy in the prime of its life for a relatively longer time.

The term “quality of life” means the ability to enjoy normal life activities.

The term “health and/or wellness of an animal” means the complete physical, mental, and social well being of the animal, not merely the absence of disease or infirmity.

As used herein, ranges are used herein in shorthand, to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a compound” or “a method” includes a plurality of such “compounds” or “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context.

The terms “comprising” or “including” are intended to include embodiments encompassed by the terms “consisting essentially of” and “consisting of”. Similarly, the term “consisting essentially of” is intended to include embodiments encompassed by the term “consisting of”.

All percentages expressed herein are by weight of the composition on a dry matter basis unless specifically stated otherwise. The skilled artisan will appreciate that the term “dry matter basis” means that an ingredient's concentration in a composition is measured after any free moisture in the composition is removed.

The methods and compositions and other advances disclosed here are not limited to particular methodology, protocols, ingredients, components and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### THE INVENTION

In one aspect, the invention provides methods for promoting sleep in an animal. The methods comprise administering in conjunction a sleep promoting amount of astaxanthin and a sleep promoting amount of melatonin to the animal. In another aspect, the methods further comprise administering in conjunction a sleep promoting amount of zinc to the animal.

The astaxanthin, melatonin, and optional zinc (i.e., the “sleep promoting ingredients”) can be administered to the animal together or in conjunction in various combinations. Administration can be on an as-needed or as-desired basis of varying or regular frequency. A goal of regular administration is to provide the animal with a regular and consistent dose of the composition or the direct or indirect metabolites that result from such administration. Such regular and consistent dosing will tend to create constant blood levels of the components of the compositions or their direct or indirect metabolites. Thus, regular administration can be once monthly, once weekly, once daily, or more than once daily. Similarly, administration can be every other day, week, or month, every third day, week, or month, every fourth day, week, or month, and the like. Administration can be multiple times per day. In a preferred embodiment, the astaxanthin, melatonin, and optional zinc are administered daily.

In another aspect, the invention provides a method for promoting the health or wellness of an animal. The method comprises administering in conjunction a health or wellness promoting amount of a combination of astaxanthin and melatonin to the animal. In another aspect, the method further comprises administering in conjunction a health or wellness promoting amount of zinc to the animal.

In an alternative aspect, the invention provides a method for improving the quality of life of an animal. The method comprises administering in conjunction a quality of life improving amount of a combination of astaxanthin and melatonin to the animal. In another aspect, the method further comprises administering in conjunction a quality of life improving amount of zinc to the animal.

In yet another aspect, the invention provides a method for extending the prime years of an animal's life. The method comprises administering a composition comprising astaxanthin and melatonin to the animal in an amount effective for extending the prime years of the animal. In another aspect, the method further comprises administering in conjunction an amount of zinc in an amount effective for extending the prime years of the animal.

In various embodiments, the astaxanthin and the melatonin can be administered with a meal. For example, the astaxanthin and the melatonin can be administered in a liquid beverage. When utilized as a supplement to ordinary dietetic requirements, the sleep promoting ingredients can be administered directly to the animal, e.g., orally. The sleep promoting ingredients can alternatively be contacted with, or admixed with, daily foods or beverages, including a fluid such as drinking water.

In various embodiments, the astaxanthin and the melatonin can be administered at any suitable time or schedule, for example, the astaxanthin and the melatonin can be administered at a time in the afternoon, in the evening or before a typical bedtime of the animal. Administration can also be carried out as part of a dietary regimen for the animal. For example, a dietary regimen may comprise regular ingestion by the animal of any compound or composition described herein in an amount effective for promoting sleep. Preferably, the compounds or compositions are administered to the animal in the evening, e.g., between 3 PM and 10 PM, and/or can be available throughout the evening and overnight.

In various embodiments, the astaxanthin can be administered in an amount of from about 0.1 to about 60 mg/kg/bw including about 5, 10, 15, 20, 25, 30, 35, 40, 45, 50, 55 mg/kg/bw and the like and any ranges in between. In various embodiments, the melatonin can be administered in an amount ranging from about 0.1 to about 40 mg/kg/bw including about 5, 10, 15, 20, 25, 30, 35 mg/kg/bw and the like. In various embodiments, the astaxanthin can be administered in an amount of from about 0.1 to about 20 mg/day and the melatonin is administered in an amount of from about 0.1 to about 30 mg/day. In various embodiments, the melatonin is administered in an amount of about 0.5, 1, 2, 3, 4, 5, 6, 7, 8, 10, 12, 14, 16, 18, and 20 mg/day. Similarly, in various embodiments, the astaxanthin is administered in an amount of about 0.5, 1, 2, 3, 4, 5, 6, 7, 8, 10, 12, 14, 16, 18, 20, 22, 24, 26, 28, and 30 mg/day.

In various embodiments, the zinc can be administered in an amount of from about 10 to about 100 mg/kg/bw. In various embodiments, the zinc can be administered in an amount of from about 10 to about 100 mg/day. In some embodiments, the zinc is administered to the animal in amounts of from about 0.5 to about 5 times the recommended daily allowance.

Administration of the compounds or compositions described herein, including administration us part of a dietary regimen, can span a period ranging from parturition through the adult life of the animal. In certain embodiments, the animal is a young or growing animal. In preferred embodiments, the animal is an aging animal or senior animal. In various embodiments, the animal can be susceptible to or suffering from a condition that impairs normal sleep, for example, the condition can be jetlag, insomnia, pain, a sleep disorder, etc.

In various embodiments, the method can further comprise administering a sleep aid drug in conjunction with the astaxanthin and the melatonin. In various embodiments, the method can further comprise administering a holistic therapy in conjunction with the astaxanthin and the melatonin.

In another aspect, the invention provides a method for promoting sleep in an animal. The method comprises administering in conjunction a sleep promoting amount of astaxanthin and one or more metabolizable compounds that can be metabolized to produce melatonin to the animal. The method can further comprise administering in conjunction a sleep promoting amount of zinc to the animal.

The metabolizable compounds are converted to melatonin by the metabolic processes in the animals, and the melatonin is involved in promoting sleep as described herein. In preferred embodiments, the metabolizable compounds are serotonin, tryptophan, 5-hydorxytryptophan or a combination thereof. The metabolizable compounds can be administered in any amount sufficient to obtain the melatonin amounts required herein upon metabolism. Typically, the metabolizable compounds are administered in a composition comprising astaxanthin and the metabolizable compounds.

In an alternative aspect, the invention provides a sleep promoting composition suitable for promoting sleep in an animal. The sleep promoting composition includes a sleep promoting amount of astaxanthin and a sleep promoting amount of melatonin combined in the same composition. The melatonin can range from about 0.1 to about 30 mg including about 1, 2, 3, 4, 5, 6, 7, 8, 9, 10, 11, 12, 13, 14, 15, 16, 17, 18, 19, 20, 21, 22, 23, 24, 25, 26, 27, 28, 29 mg and the like and any ranges in between. The astaxanthin can range from about 0.1 to about 20 mg including about 1, 2, 3, 4, 5, 6, 7, 8, 9, 10, 11, 12, 13, 14, 15, 16, 17, 18, 19 mg and the like and any ranges in between. The sleep promoting composition can further include zinc ranging from about 10 to about 100 mg.

In another aspect, the invention provides a sleep promoting composition suitable for promoting sleep in an animal. The composition includes a sleep promoting amount of astaxanthin and one or more metabolizable compounds that can be metabolized to produce melatonin in the animal. The metabolizable compound can be serotonin, tryptophan, 5-hydorxytryptophan or a combination thereof. The sleep promoting composition can further include zinc ranging from about 10 to about 100 mg.

The sleep promoting compositions in any embodiments described herein can be, for example, in the form of a snack, a beverage, a pet food composition, a dietary supplement, a pharmaceutical dosage form, etc. The sleep promoting compositions can include any suitable ingredients or components suitable for administering.

The sleep promoting compositions can include ingredient such as, for example, proteins, preservatives, oral care ingredients, visible nutrition, colorants, flavorants, humectants, antioxidants, vitamins, minerals or a combination thereof in any suitable amounts. The additional ingredients can further by using to promote a healthy sleep and healthy lifestyle of the animal.

The protein may be derived from a plant or animal source or both. It may be provided as a protein concentrate. Suitable examples of preservatives include potassium sorbate, sorbic acid, methyl parahydroxybenzoate, calcium propionate and propionic acid.

The oral care ingredients can provide breath freshening and/or tartar control. Suitable oral care ingredients include alfalfa nutrient concentrate (contains chlorophyll), sodium bicarbonate, phosphates (e.g., tricalcium phosphate, acid pyrophosphates, tetrasodium pyrophosphate, metaphosphates, orthophosphates), peppermint, cloves, parsley, ginger, etc.

The visible nutrition ingredients can be in the form of pieces or specks on the surfaces and/or within the sleep promoting compositions. Suitable visible nutrition ingredients include corn germ meal, dehydrated vegetables, fruits, grains (e.g., spinach, carrots, cranberry), etc.

The colorants can provide an aesthetic effect. Suitable colorants include FD & C colors, natural colors, titanium dioxide, etc. The flavorants can make the multi-textured treat more palatable for the animal. Suitable flavorants include yeast, tallow, rendered animal meals (e.g., poultry, beef, lamb, pork), flavor extracts or blends (e.g., grilled beef), etc.

Suitable humectants include salt, sugars, propylene glycol and polyhydric glycols such as glycerin and sorbitol, and the like. Suitable antioxidants include BHA/BHT, vitamin E (tocopherols), etc.

Suitable vitamins may include Vitamins A, B-complex (such as B-1, B-2, B-6 and B-12), C, D, E and K, niacin and acid vitamins such as pantothenic acid and folic acid and biotin. Suitable minerals may include calcium, iron, zinc, magnesium, iodine, copper, phosphorus, manganese, potassium, chromium, molybdenum, selenium, nickel, tin, silicon, vanadium and boron.

In another aspect, the invention provides a kit suitable for promoting sleep or a healthy lifestyle in an animal. The kit includes in separate containers in a single package or in separate containers in a virtual package, as appropriate for the kit component, one or more of the sleep promoting compositions in any embodiments described herein and one or more of (1) a comestible product to be taken in conjunction with the sleep promoting composition, (2) a beverage to be taken in conjunction with the sleep promoting composition, (3) instructions for how to promote sleep in an animal using the sleep promoting composition, (4) instructions for how to promote sleep in an elderly animal using the sleep promoting composition, (5) instructions for how to promote the health or wellness of an animal using the sleep promoting composition, (6) instructions for how to improve the quality of life of an animal using the sleep promoting composition, (7) instructions for how to extend the prime years of an animal's life using the sleep promoting composition, or (8) a sleep toy for an animal. The instructions can be suited for specific types of animals as well (e.g., cats, dogs, or humans).

When the kit comprises a virtual package, the kit can be limited to instructions in a virtual environment in combination with one or more physical kit components. The kits may contain the kit components in any of various combinations and/or mixtures. For example, in one embodiment, the kit contains a package containing the sleep promoting composition and a comestible product to be taken in conjunction with the sleep promoting composition. In another embodiment, the kit contains a package containing the sleep promoting composition and instructions for how to promote sleep in an animal using the sleep promoting composition.

In another aspect, the invention provides a means for communicating information about or instructions for using the sleep promoting composition in any embodiments described herein for one or more of (1) promoting sleep in an animal; (2) promoting sleep in an elderly animal; (3) promoting the health or wellness of an animal; (4) improving the quality of life of an animal; or (5) extending the prime years of an animal's life, the means comprising a document, digital storage media, optical storage media, audio presentation, or visual display containing the information or instructions.

The communication means can be a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, or any combination thereof. The communication means is useful for instructing on the benefits of using the sleep promoting compositions and communicating about the approved methods for using the sleep promoting compositions for an animal.

In another aspect, the invention provides a use of astaxanthin and melatonin to prepare a sleep promoting composition useful for promoting sleep in an animal. In certain embodiments, the sleep promoting composition further comprises zinc.

In another aspect, the invention provides an animal food package comprising a container and a plurality of sleep promoting compositions stored within the container. The compositions comprising a sleep promoting amount of astaxanthin and a sleep promoting amount of melatonin. The package can further include a label affixed to the package containing a word or words, picture, design, acronym, slogan, phrase, or other device, or combination thereof, that indicates that the contents of the package contains the sleep promoting compositions (e.g., information about the sleep promoting compositions and/or its physical, functional, and related properties).

Typically, such device can include the words “sleep promoting compositions for animals” or an equivalent expression printed on the package. Any package or packaging material suitable for containing sleep promoting compositions is useful in the invention, e.g., a bag, box, bottle, can, pouch, and the like manufactured from paper, plastic, foil, metal, and the like.

In another aspect, the invention provides a method of making a sleep promoting composition. The method comprises adding a sleep promoting amount of astaxanthin and a sleep promoting amount of melatonin to a comestible composition using any suitable manufacturing process known in the art.

In a further aspect, the invention provides for a use of a sleep promoting amount of astaxanthin and a sleep promoting amount of melatonin to prepare a medicament useful for promoting sleep, promoting the health or wellness of an animal, improving the quality of life of an animal, and extending the prime years of an animal's life. Generally, medicaments are prepared by admixing the compounds with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal. In one embodiment, the medicament further comprises zinc. The medicaments contain the compounds in amounts specified herein, e.g., from about 0.1 to about 60 mg/kg/bw astaxanthin, from about 0.1 to about 40 mg/kg/bw melatonin, and from about 10 to about 100 mg/day zinc.

In another aspect, the invention provides a package useful for containing a sleep promoting amount of astaxanthin and a sleep promoting amount of melatonin, and optionally a sleep promoting amount of zinc. The package comprises at least one material suitable for containing the compounds and a label affixed to the material containing a word or words, picture, design, acronym, slogan, phrase, or other device, or combination thereof, that indicates that the package contains the compounds and/or their function, e.g., promoting sleep. Typically, such device comprises the words “promotes sleep” or “contains natural sleep promoting compounds” or an equivalent expression printed on the material. Any package configuration and packaging material suitable for containing compounds are useful in the invention, e.g., a bag, box, bottle, can, pouch, and the like manufactured from paper, plastic, foil, metal, and the like. In various embodiments, the package further comprises at least one window that permit the package contents to be viewed without opening the package. In some embodiments, the window is a transparent portion of the packaging material. In others, the window is a missing portion of the packaging material.

### EXAMPLES

The invention can be further illustrated by the following examples, although it will be understood that these examples are included merely for purposes of illustration and are not intended to limit the scope of the invention unless otherwise specifically indicated.

### Example 1

Forty eight (48) dogs that were at least 9 years of age were placed into treatment and control groups after the collection of baseline data from all the animals. Each group contained 24 animals. The animals were fed either (1) a control food containing 28% crude protein, 12% crude fat, 3% crude fiber, and 181 ppm zinc or (2) a treatment food containing 28% crude protein, 12% crude fat, and 3% crude fiber and 13.5 ppm melatonin, 8.2 ppm astaxanthin, and 208 ppm zinc. The amount of melatonin was based upon a dose of about 0.1 mg/kg/bw.

The animals were administered the control or treatment food for 77 days. All animals were fed twice daily, at 9 AM (±30 minutes) and 6 PM (±30 minutes). The animals in the treatment group were fed the control food with the morning feeding and the treatment food with the afternoon feeding. Animals assigned to the control group were led twice daily, a 9 AM (±30 minutes) and 6 PM (±30 minutes), and were fed the control food in the AM and PM.

### Example 2

Using a cross-over design, all animals were administered a control placebo and a treatment supplement containing melatonin. The animals were senior animals, dogs greater than 10 years of age. There were 4 males and 4 females in each group. For the intervention phases (phases 2 and 4), all animals were administered melatonin in the AM or PM. During the control and cross-over phases (phase 1 and 3, respectively) the animals were administered a placebo comprising methylcellulose contained in a gel capsule in the AM or PM, as appropriate.

Each phase lasted 35 days. Starting on day 29 of each phase, the animals locomotor behavioral activity was monitored for 4 consecutive days.

The placebo consisted of the carrier (methylcellulose) contained in a gel capsule and was provided during the control phases (Phases 1 and 3) either with the AM or PM feeding, as appropriate to complement the supplement time (luring the following intervention phase. For example, placebo in AM during phase 1, then supplement in AM during phase 2.

For phase 2, the first supplement treatment phase, the animals were given supplements of melatonin. Further, for half of the animals, the supplements were in the morning with the AM feeding and half were given in the evening with the PM feeding. Thus, of the 4 males, 2 were administered the melatonin in the morning and two in the evening.

During phase 3, the second control phase, delivery of the methylcellulose in a gel capsule was provided either with the AM or PM feeding, as appropriate to cross-over the treatment from phase 1-2.

For phase 4, the second supplement treatment phase, the procedure was exactly the same as phase 2, except that for a given animal the supplementation time was switched from phase 2. Thus, if male was administered melatonin in the morning with the AM feeding in phase 2, the male was administered melatonin in the evening with the PM feeding in phase 4.

Data analysis of periods indicated no period effect. Therefore, all control data were compiled into a single dataset, as well as all treatment data for each supplement time (AM or PM). Statistical analysis was based on AM control vs. AM treatment, and PM control vs. PM Treatment.

### Example 3

Potty eight (48) dogs that were at least 9 years of age were placed into treatment and control groups after the collection of baseline data from all the animals. Bach group contained 24 animals. The animals were led either (1) a control food containing 26% crude protein, 16% crude fat, and 3% crude fiber or (2) a treatment food containing 26% crude protein, 16% crude fat, and 3% crude fiber and 130 ppm melatonin. The amount of melatonin was based upon a dose of about 0.1 mg/kg/bw. The animals were administered the control or treatment food for 77 days. All animals were fed twice daily, at 9 AM (±30 minutes) and 6 PM (±30 minutes). Animals in the control and treatment groups differed only by supplementation of placebo or melatonin.

### Procedures and Data Recorded

### A. Activity Recording

Twenty-four hour activity rhythms were assessed for 5 consecutive days and 5 nights for Example 1 or 3 consecutive days and 4 nights for Example 2 using the Actiwatch method (Mini-Mitter® Actiwatch-16® activity monitoring system, Respironics Co. Inc., Bend, Oreg.). The Actiwatch® was placed inside a specially designed animal case and attached to a collar around the dog's neck. The dogs were allowed to follow their usual patterns of activity, test, exercise, and feeding.

For Example 1, activity data was recorded during the baseline phase of the Example when all dogs were consuming only the control food. After 65 days of the treatment phase, in which dogs were either on the control or treatment foods, the animals were monitored again for locomotor activity behavior patterns by recording activity data. The monitors recorded activity counts on a 30-sec epoch setting and activity data was downloaded to a PC-computer immediately following the completion of the data recording period for later analysis. Total daily, light phase (day), and dark phase (night) activity counts were generated by the Actiware® software provided with the Actiwatch® recording system, along with dark/light phase activity counts ratio.

### B. Sleep Analysis

The Actiware® software was used to generate total daily, light phase (12-h daytime: 7 am-7 pm), and dark phase (12-h night-time: 7 PM-7 AM) total sleep or wake minutes. Light phase naps represent sleep bouts occurring during the 12-h light phase interval. Dark phase wake bouts relied awake activity during the night-time sleep interval and not the 12-h dark phase interval. Total number of bouts and bout duration were determined for both light phase naps and dark phase awakenings.

### Activity Recordings

### Daytime Activity and Diet Effect with Melatonin, Astaxanthin, and Zinc

Daytime activity was recorded to represent the 12 hour light phase from 7 am to 7 pm. Senior dogs over the age of 9 yrs old consumed the PM diet enriched with melatonin (0.1 mg/kg/bw), approximately 1 mg daily dose), astaxanthin and zinc for 65 days and had a 30% reduction in total daytime activity counts compared to dogs on the control food (Table 1). This was also a 30% reduction in daytime activity counts compared to baseline levels in the test group, whereas activity counts changed less than 1% from baseline levels in the control group.

TABLE 1
Mean +/− Std Error activity of control group dogs (n =
23) compared to test dogs (n = 22) (Example 2)

| | Total Daytime Activity Counts | |
|---|---|---|
| | Baseline Phase | Treatment Phase |
| Control Food Group | 228,115 +/− 19,992 | 226,624 +/− 39,128 |
| Test Food Group With | 227,929 +/− 26,093 | 158,856 +/− 21,119 |
| Melatonin, Astaxanthin, | | |
| and Zinc | | |

### Daytime Activity and Diet Effect with Melatonin Only with PM Meal

Daytime activity was recorded to represent the 12 hour light phase from 7 am to 7 pm. Senior dogs over the age of 10 yrs old consumed the PM diet enriched with only melatonin (1 mg/kg BW, approximately 10 mg daily dose) for 35 days and was observed to only have a 16% reduction in total daytime activity counts compared to dogs on the control food (Table 2). This effect was not statistically significant and the daily dose of melatonin was approximately 10 times higher than the study with melatonin combined with astaxanthin and zinc. A similar effect was observed when melatonin was supplemented in the AM, and was also not significantly different from the placebo group.

TABLE 2
Mean +/− Std Error activity of control dogs (n =
23, no melatonin) compared to test group dogs (n = 22)
fed a test diet in the evening enriched with only melatonin (Example 2)

| | Total Daytime Activity Counts | |
|---|---|---|
| | AM Supplement | PM Supplement |
| Control Food Group | 138,875 +/− 38,459 | 136,580 +/− 28,762 |
| Test Food Group With | 115,705 +/− 23,249 | 114,816 +/− 34,282 |
| Melatonin Only | | |

### Daytime Sleep Minutes and Diet Effect with Melatonin, Astaxanthin, and Zinc

Daytime activity was used to estimate the total number of minutes recorded above a pre-selected activity threshold that predicts the animal is awake at each 60 sec recording epoch. Correspondingly, epochs not determined to be above the threshold are categorized as the animal being asleep during that 60 sec epochs. The total minutes of sleep during the 12 hour daytime phase increased by 17.7% from baseline with the test group, whereas total minutes of sleep for the control group were only approximately 3% different from baseline levels (Table 3).

TABLE 3
Mean +/− Std Error total daytime sleep minutes
for control group dogs (n = 23) compared to
test group dogs (n = 22) (Experiment 1).

Total Daytime Sleep Minutes

| | Baseline Phase | Treatment Phase |
|---|---|---|
| Control Food Group | 251.7 +/− 13.0 | 259.6 +/− 11.8 |
| Test Food Group With | 240.9 +/− 17.1 | 283.6 +/− 18.5 |
| Melatonin, Astaxanthin, | | |
| and Zinc | | |

### Daytime Sleep Minutes and Diet Effect with Melatonin Only

Senior dogs over the age of 10 yrs old consumed the PM diet enriched with only melatonin (1 mg/kg BW, approximately 10 mg daily dose) for 35 days and had a 21% increase in total daytime sleep minutes compared to dogs on the control food (Table 4). This was not statistically significant (p=0.25)

TABLE 4
Mean +/− Std Error activity of control dogs (n =
23, no melatonin) compared to test group dogs (n = 22)
fed a test diet in the evening enriched with only melatonin (Experiment 2)

Total Daytime Sleep Minutes

| | AM Supplement | PM Supplement |
|---|---|---|
| Control Food Group | 282 +/− 28 | 217 +/− 28 |
| Test Food Group With | 240 +/− 32 | 264 +/− 28 |
| Melatonin Only | | |

### Daytime Naps and Nap Duration and Diet Effect with Melatonin, Astaxanthin, and Zinc

The total number of sleep minutes recorded during the daytime phase was used to calculate the number of nap bouts and average nap duration during this same period. The total number of naps during the 12 hour daytime phase increased by 17.2% from baseline with the test group, whereas total minutes of sleep for the control group were only approximately 6% different from baseline levels (Table 5). In addition, nap duration increased by an average of 16.4% from baseline with the test group, whereas the control group had shorter naps by approximately 11% from baseline levels (Table 5).

TABLE 5
Mean +/− Std Error total daytime naps for control group dogs (n =
23) compared to test group dogs (n = 22) (Example 1).

| | Total Daytime Naps | | Nap Duration | |
|---|---|---|---|---|
| | Baseline | Treatment | Baseline | Treatment |
| | Phase | Phase | Phase | Phase |
| Control | 57.8 +/− 3.0 | 61.5 +/− 2.2 | 4.5 +/− 0.3 | 4.0 +/− 0.2 |
| Food Group | | | | |
| Test Food | 57.4 +/− 3.0 | 67.2 +/− 3.2 | 4.2 +/− 0.3 | 4.9 +/− 0.3 |
| Group With | | | | |
| Melatonin, | | | | |
| Astaxanthin, | | | | |
| and Zinc | | | | |

### Daytime Naps and Nap Duration and Diet Effect with Melatonin Alone with PM Meal

The total number of sleep minutes recorded during the daytime phase of Example 2 was used to calculate the number of nap bouts and average nap duration during this same period. The total number of naps during the 12 hour daytime phase did not change at all with the test group supplemented with melatonin with the PM meal compared to the control group. In contrast, melatonin supplemented with the AM meal resulted in a 10% increase in the number of naps compared to control group (Table 6).

The average nap duration daring the 12 hour daytime phase increased by 18.5% with the test group supplemented with melatonin with the PM meal compared to the control group. This increase in nap duration is the reason why total daytime sleep minutes increased (Table 4). In contrast, melatonin supplemented with the AM meal resulted in a 31% decrease in average nap length compared to control group (Table 6). Similarly, this decrease in nap duration can be attributed to the quantitative decrease in total daytime sleep minutes with AM melatonin supplement (Table 4).

TABLE 6
Mean +/− Std Error total daytime naps for control group dogs (n =
23) compared to test group dogs (n = 22) (Example 2).

| | Total Daytime Naps | | Nap Duration | |
|---|---|---|---|---|
| | AM | PM | AM | PM |
| | Supple- | Supple- | Supple- | Supple- |
| | ment | ment | ment | ment |
| Control | 53.1 +/− 4.2 | 45.4 +/− 4.1 | 6.1 +/− 0.6 | 5.4 +/− 1.3 |
| Food Group | | | | |
| Test Food | 58.3 +/− 3.5 | 45.5 +/− 4.7 | 4.2 +/− 0.7 | 6.4 +/− 1.2 |
| Group With | | | | |
| Melatonin Only | | | | |

### Total Night-Time Wake Minutes and Diet Rifled with Melatonin, Astaxanthin, and Zinc

The total number of wake minutes recorded during the 12-h night phase was estimated similarly to the total daytime sleep minutes using the night-time activity count data. The total night-time wake minutes during the 12 hour nighttime phase increased by 12.6% from baseline with the test group, whereas total minutes of wake time for the control group was only approximately 6% different from baseline levels (Table 7).

TABLE 7
Mean +/− Std Error total night-time wake minutes for control group dogs
(n = 23) compared to test group dogs (n = 22) (Example 1).

Total Night-Time Wake Minutes

| | Baseline Phase | Treatment Phase |
|---|---|---|
| Control Food Group | 147 +/− 8 | 138 +/− 6 |
| Test Food Group With | 157 +/− 8 | 137 +/− 13 |
| Melatonin, Astaxanthin, | | |
| and Zinc | | |

### Total Night-Time Wake Minutes and Diet Effect with Melatonin Alone

The total number of night-time wake minutes during the 12 hour night-time phase of Example 2 decreased by 18.6% with the test group supplemented with melatonin with the PM meal compared to the control group. However, this difference was nut statically significant. In contrast, melatonin supplemented with the AM meal resulted in a 41% increase in night-time wake minutes compared to control group (Table 8). Similarly, test group data was not different from control data.

TABLE 8
Mean +/− Std Error total night-time wake minutes for control group dogs
(n = 23) compared to test group dogs (n = 22) (Example 2).

Total Night-Time Wake Minutes

| | AM Supplement | PM Supplement |
|---|---|---|
| Control Food Group | 141 +/− 43 | 257 +/− 41 |
| Test Food Group With | 198 +/− 35 | 209 +/− 37 |
| Melatonin Only | | |

### Night-Time Awakenings and Diet Effect with Melatonin, Astaxanthin, and Zinc

The total number of wake minutes recorded during the night-time phase was used to calculate the number of wake bouts and average nap duration during this same period. Wake bout duration did not differ with diet. The total number of night-time awakenings during the night-time sleep phase (actual phase when animals are considered asleep at night) decreased by 2% from baseline with the test group, whereas the control group increased the number of awakenings by 3% from baseline levels (Table 9). Additionally, wake bouts differed by 9.4% with test diet compared to control diet during the treatment phase (Table 9).

TABLE 9
Mean +/− Std Error total night-time awakenings for control group dogs
(n = 23) compared to test group dogs (n = 22) (Example 1).

Total Night-Time Awakenings

| | Baseline Phase | Treatment Phase |
|---|---|---|
| Control Food Group | 51.2 +/− 2.5 | 52.0 +/− 2.9 |
| Test Food Group With | 48.7 +/− 3.3 | 47.8 +/− 2.9 |
| Melatonin, Astaxanthin, | | |
| and Zinc | | |

### Daytime Naps and Nap Duration and Diet Effect with Melatonin Alone with PM Meal

When melatonin was supplemented alone with the PM meal, the total number of awakenings during the sleep phase decreased by 22% with the test group compared to the control group. In contrast, melatonin supplemented with the AM meal resulted in a 16.6% increase in the number of awakenings compared to control group (Table 10).

TABLE 10
Mean +/− Std Error total daytime naps for control group dogs (n =
23) compared to test group dogs (n = 22) (Example 2).

Total Night-Time Awakenings

| | AM Supplement | PM Supplement |
|---|---|---|
| Control Food Group | 59.6 +/− 5.8 | 67.5 +/− 6.5 |
| Test Food Group With | 69.5 +/− 6.9 | 52.6 +/− 5.7 |
| Melatonin Only | | |

Referring to the data, the results show that the combination of melatonin, astaxanthin, and zinc provided beneficial effects above the use of only melatonin. Particularly notable was that daytime activity was reduced. When activity data was used to estimate the daytime sleeping patterns, the analysis showed that the animal was experiencing more daytime naps and slightly longer daytime naps. This effect was not observed when only melatonin was supplemented in the evening, as resulting daytime activity did not significantly decline and the number of daytime naps did not change.

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims the invention may be practiced otherwise than as specifically described.

1. A method for promoting sleep in an animal comprising administering in conjunction a sleep promoting amount of astaxanthin and a sleep promoting amount of melatonin to the animal.

2. The method of claim 1 wherein the astaxanthin is administered in an amount of from about 0.1 to about 60 mg/kg/bw and the melatonin is administered in an amount of from about 0.1 to about 40 mg/kg/bw.

3. The method of claim 1 wherein the astaxanthin is administered in an amount of from about 0.1 to about 20 mg/day and the melatonin is administered in an amount of from about 0.1 to about 30 mg/day.

4. The method of claim 1 further comprising administering in conjunction a sleep promoting amount of zinc to the animal.

5. The method of claim 4 wherein the zinc is administered in an amount of from about 10 to about 00 mg/kg/bw.

6. The method of claim 4 wherein the zinc is administered in an amount of from about 10 to about 100 mg/day.

7. (canceled)

8. (canceled)

9. (canceled)

10. (canceled)

11. The method of claim 1 wherein the animal is a senior animal.

12. The method of claim 1 wherein the animal is susceptible, to or suffering from a condition that impairs normal sleep.

13. (canceled)

14. (canceled)

15. (canceled)

16. (canceled)

17. The method of claim 1 further comprising administering a sleep aid drug in conjunction with the astaxanthin and the melatonin.

18. The method of claim 1 further comprising administering a holistic therapy in conjunction with the astaxanthin and the melatonin.

19. A method for promoting sleep in an animal comprising administering in conjunction a sleep promoting amount of astaxanthin and one or more metabolizable compounds that can be metabolized to produce melatonin to the animal.

20. The method of claim 19 further comprising administering in conjunction a sleep promoting amount of zinc to the animal.

21. The method of claim 19 wherein the metabolizable compound is selected from the group consisting of serotonin, tryptophan, 5-hydorxytryptophan and combinations thereof.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A use of astaxanthin and melatonin to prepare a sleep promoting composition useful for promoting sleep in an animal.

41. The use of claim 40 wherein the sleep promoting composition further comprises a sleep promoting amount of zinc.

42. (canceled)

43. (canceled)

44. (canceled)

45. A use of a sleep promoting amount of astaxanthin and a sleep promoting amount of melatonin to prepare a medicament for one or more of promoting sleep, promoting the health or wellness of an animal, improving the quality of life of an animal, and extending the prime years of an animal's life.

46. The medicament of claim 45 further comprising a sleep promoting amount of zinc.

47. (canceled)

48. (canceled)

49. (canceled)

50. (canceled)

51. (canceled)

52. (canceled)
